(19) United States
(12) Patent Application Publication
(10) Pub. No.: US 2026/0308508 A1
(43) Pub. Date: Oct. 8, 2026

(54) PROCESS FOR PRODUCING MICROALGAE OIL AND VALUABLE PRODUCTS FROM WET MICROALGAE BIOMASS

(71) Applicant: National Key Laboratory for Petrochemical and Refinery Technologies, Hanoi (VN)

(72) Inventor: Thu Ha Thi Vu, Hanoi (VN)

(21) Appl. No.: 19/355,591

(22) Filed: Oct. 10, 2025

(30) Foreign Application Priority Data
Apr. 8, 2025 (VN) 1-2025-02365

(51) Int. Cl. C12N 1/12; C12N 13/00

(52) U.S. Cl. CPC C12N 1/12; C12N 13/00

(57) ABSTRACT

A process of producing microalgae oil from wet microalgae biomass includes steps of: bead milling to preliminarily break cell walls; ultrasonic treatment and mixing in a device equipped with ultrasonic emitters to physically disrupt cell walls, combined with enzymatic autolysis by endogenous enzymes; followed by primary and secondary centrifugation to extract oil and valuable compounds; then centrifuging in a conical centrifuge to obtain microalgae oil and a water phase containing valuable water-soluble compounds, and finally concentrating this water phase to yield valuable water-soluble products.

### CROSS-REFERENCE TO RELATED APPLICATION

This application claims priority from Vietnamese Application No. 1-2025-02365 filed Apr. 8, 2025, which is incorporated herein by reference in its entirety.

### TECHNICAL FIELD

The invention relates to a process of producing microalgae oil from wet microalgae biomass. More specifically, the invention relates to a process of directly extracting lipids from wet microalgae biomass performed at low temperatures without the use of chemicals, solvents, or exogenous enzymes to obtain pure microalgae oil and accompanying valuable by-products.

### RELATED ART

One of the humanity's primary goals is to ensure the sustainability of food systems, including the management of land, water, and biodiversity. Therefore, microalgae are considered a viable and sustainable alternative raw material due to their rich chemical compositions. Particularly regarding lipids, microalgae can synthesize and accumulate significant amounts of fatty acids, mostly polyunsaturated fatty acids, making them excellent candidates for lipid production and extraction in various industrial and medical sectors, either as bulk products or pure chemicals.

Microalgae can produce fatty oils year-round. The fatty oil productivity of microalgae is higher than that of conventional crops. The lipid (fatty oil) content of microalgae ranges from 20% to 50%, which is higher than other competing biomass sources. Microalgae can grow at a high rate, 50 times as fast as switchgrass, the fastest-growing terrestrial crop. Microalgae have a higher photon conversion efficiency than all crops, reaching values of approximately 3% to 8% compared to 0.5% for terrestrial plants. Microalgae are an alternative fuel source that does not compete with food, and they can be cultivated in wastewater as a nutrient source. Industrial microalgae cultivation processes are integrated processes for CO₂ capture, contributing to solving global warming and generating valuable by-products such as lipids, carbohydrates, proteins, and various other raw materials that can be converted into biofuels and other useful materials. Microalgae have the potential to become an essential raw material source for producing amino acids, vitamins, and generating valuable by-products. Microalgae production is known to be a more profitable business in biotechnology. It is a low-waste and environmentally safe process.

Therefore, the cultivation and pre-processing of microalgae biomass to obtain preparations as raw materials for deeper processing, yielding high added value, are always of particular interest. However, due to the very rigid cell walls of most microalgae species feasible for industrial-scale cultivation, breaking the cell walls to improve the efficiency of component separation from microalgae biomass is one of the significant challenges to overcome. Specifically, except for Schizochytrium sp., which has a relatively soft cell wall (with no cellulose layer in its cell wall, only containing phospholipid membranes), most other microalgae species currently being developed at an industrial scale have rigid and durable cell walls. For example, the cell wall of the genus Nannochloropsis sp. contains cellulose, glycoproteins, and silica; the genus Chlorella sp. contains chitin and cellulose; and the species Chlorella sorokiniana contains cellulose and glycoproteins.

Cell disruption of microalgae is considered a crucial pre-treatment step in the extraction of oil and other compounds from microalgae biomass. The most important factors determining cell wall strength are its thickness, structure, and chemical composition, which impart unique characteristics to the cells of different microalgae species. Therefore, cell wall characteristics are very important in determining the most effective disruption methods. Cell disruption methods can be divided into two types: mechanical and non-mechanical (André Luiz Figueiredo Nunes; Viviane Sobreira Lima1 José Roberto Miranda Júnior; Monique Ellen Torres Resende; César Augusto Sodré da Silval Márcio Arêdes Martins; Jane Selia dos Reis Coimbra, Cell disruption of microalgae: advances and perspectives Ciência Rural, Santa Maria, v. 54:05, e20220330, 2024). Mechanical methods include high-pressure homogenization, milling, ultrasound, microwave, and pulsed electric field techniques. Non-mechanical methods include techniques such as chemical, thermal, and enzymatic disruption.

### High-Pressure Homogenization (HPH):

In this technique, cells are subjected to shear stress that promotes cell wall disruption, facilitating the separation of intracellular compounds. The disadvantage of HPH is that it causes non-selective release of intracellular components. It can produce high concentrations of cell debris, hindering the subsequent separation step and causing undesirable temperature increases for heat-sensitive extracts such as enzymes, lipids, pigments, and proteins (Angles, E. et al. Wet lipid extraction from the microalga Nannochloropsis sp. disruption, physiological effects and solvent screening. Algal Research, v. 21, p. 27-34, 2017; Gul, O. et al. Effect of high-pressure homogenization (HPH) on microstructure and rheological properties of hazelnut milk. Innovative Food Science & Emerging Technologies, v. 41, p. 411-420, 2017; Lee, S. Y. et al. Cell disruption and lipid extraction for microalgal biorefineries: A review. Bioresource Technology, v. 244, n. 2, p. 1317-1328, 2017; Spiden, E. M. et al. Quantitative evaluation of the ease of rupture of industrially promising microalgae by high pressure homogenization. Bioresource Technology, v. 140, p. 165-71, 2013; Günerken, E. et al. Cell disruption for microalgae biorefineries. Biotechnology Advances, v. 33, n. 2, p. 243-260, 2015; Bernaerts, T. M. et al. The potential of microalgae and their biopolymers as structuring ingredients in food: A review. Biotechnology Advances, v. 37, n. 8, e107419, 2019; Yap, B. H. et al. Energy evaluation of algal cell disruption by high pressure homogenization. Bioresource Technology, v. 184, p. 280-285, 2015; Grossmann, L. et al. Production of protein-rich extracts from disrupted microalgae cells: Impact of solvent treatment and lyophilization. Algal Research, v. 36, p. 67-76, 2018; Ebert, S. et al. Emulsifying properties of water-soluble proteins extracted from the microalgae: Chlorella sorokiniana and Phaeodactylum tricornutum. Food & Function, v. 10, n. 2, p. 754-64, 2019; Serive, B. et al. Selection and optimization of a method for efficient metabolites extraction from microalgae. Bioresource Technology, v. 124, p. 311-320, 2012).

### Milling Technique:

Milling is a complex mechanical cell wall disruption technique where the rotating motion of milling beads promotes friction between cells and milling beads. As a result, cells are disrupted. Mechanical collision between cells and milling beads is influenced by parameters such as biomass feed rate and density, and the mass and speed of milling beads. These parameters depend on the industrial design of the milling chamber. Zinc material is recommended for producing high-density milling beads, while glass is recommended for producing low-density milling beads. Zirconia is the most useful milling bead material for milling high-viscosity materials, while glass beads are more suitable for milling low-viscosity materials. Bead diameter and bead loading conditions can determine cell lysis efficiency (Gong, M., Bassi, A. Carotenoids from microalgae: a review of recent developments. Biotechnology Advances, v. 34, n. 8, p. 1396-1412, 2016; Wang, M. et al. Algal cell lysis by bacteria: A review and comparison to conventional methods. Algal Research, v. 46, e101794, 2020; Günerken, E. et al. Cell disruption for microalgae biorefineries. Biotechnology Advances, v. 33, n. 2, p. 243-260, 2015; Schüler, L. M. et al. Improvement of carotenoid extraction from a recently isolated, robust microalga, Tetraselmis sp. CTP4 (Chlorophyta). Bioprocess and Biosystems Engineering, v. 43, p. 785-796, 2020; River A, E. C. et al. Mechanical cell disruption of Parachlorella kessleri microalgae: impact on lipid fraction composition. Bioresource Technology, v. 256, p. 77-85, 2018; Liu, S. et al. Coupling bead-milling and microfiltration for the recovery of lipids and proteins from Parachlorella kessleri: Impact of the cell disruption conditions on the separation performances. Separation and Purification Technology, v. 287, n. 15, e120570, 2022; Amorim, M. L. et al. Microalgae proteins: production, separation, isolation, quantification, and application in food and feed. Critical Reviews in Food Science and Nutrition, v. 61, n. 12, p. 1976-2002, 2021).

### Ultrasound Technique:

Among mechanical methods, the ultrasound method uses sound waves to transmit pressure fluctuations, generating bubbles and promoting cell disruption. Since this technique does not use high temperatures, it promises to extract heat-labile compounds such as proteins. However, the sonication process more or less increases the temperature of the sonicated material, so temperature also needs to be considered during the sonication process. This method is suitable for disrupting microalgae with rigid cell walls, such as Chlorella sp., but it is not indicated for microalgae strains with flexible cell walls (Do Carmo Cesário, C. et al. Biochemical and morphological characterization of freshwater microalga Tetradesmus obliquus (Chlorophyta: Chlorophyceae). Protoplasma, p. 1-12, 2021; Goh, B. H. H. et al. Sustainability of direct biodiesel synthesis from microalgae biomass: A critical review. Renewable and Sustainable Energy Reviews, v. 107, p. 59-74, 2019; Günerken, E. et al. Cell disruption for microalgae biorefineries. Biotechnology Advances, v. 33, n. 2, p. 243-260, 2015; Lee, S. Y. et al. Cell disruption and lipid extraction for microalgal biorefineries: A review. Bioresource Technology, v. 244, n. 2, p. 1317-1328, 2017; Kurokawa, M. et al. Effect of sonication frequency on the disruption of algae. Ultrasonics Sonochemistry, v. 31, p. 157-162, 2016; Safi, C. et al. Understanding the effect of cell disruption methods on the diffusion of Chlorella vulgaris proteins and pigments in the aqueous phase. Algal Research, v. 8, p. 61-68, 2015; Ido, A. L. et al. Application of central composite design in the optimization of lipid yield from Scenedesmus obliquus microalgae by ultrasound-assisted solvent extraction. Energy, v. 157, n. 15, p. 949-956, 2018; Silva, M. E. T. DA et al. Microalga Scenedesmus obliquus: extraction of bioactive compounds and antioxidant activity. Revista Ciência Agronômica, v. 52, n. 2, 2021b; Gille, A. et al. Bioaccessibility of carotenoids from Chlorella vulgaris and Chlamydomonas reinhardtii. International Journal of Food Sciences and Nutrition, v. 67, n. 5, p. 507-513, Jul. 3, 2016; Silva, M. E. T. et al. Scenedesmus obliquus protein concentrate: a sustainable alternative emulsifier for the food industry. Algal Research, v. 59, e102468, 2021a; Vieira, B. B. et al. Optimized extraction of neutral carbohydrates, crude lipids and photosynthetic pigments from the wet biomass of the microalga Scenedesmus obliquus BR003. Separation and Purification Technology, v. 269, n. 12, 2021; Lima, V. S. et al. Stabilization of oil-water emulsions with protein concentrates from the microalga Tetradesmus obliquus. Journal of Food Science and Technology, v. 60, n. 2, p. 797-808, 2023).

### Microwave Technique:

In the microwave technique, heat is transferred from the external environment to the inside of the cell. The presence of polar molecules, such as water, contributes to the rapid energy absorption of the cell. Accordingly, internal pressure increases, promoting intracellular lysis and facilitating the release of components into the extraction solvent, especially lipids. Thus, for water molecules, thermal energy is generated due to the friction of water molecules inside the cell, water evaporates, and the cell is disrupted by the pressure of water vapor on the cell wall. Since heating facilitates the extraction of lipids from microalgae and can reduce extraction time and energy consumption, microwave is a tested technique for microalgae lipid extraction. However, the heat generated by this method can affect oil quality, and this method is not yet feasible for large-scale deployment (Tigrine-Kordjani, N. et al. Contribution of microwave accelerated distillation in the extraction of the essential oil of Zygophyllum album L. Phytochemical Analysis, v. 22, n. 1, p. 1-9. 2011; Goh, B. H. H. et al. Sustainability of direct biodiesel synthesis from microalgae biomass: A critical review. Renewable and Sustainable Energy Reviews, v. 107, p. 59-74, 2019; Zhou, X. et al. Optimization of microwave assisted lipid extraction from microalga Scenedesmus obliquus grown on municipal wastewater. Journal of Cleaner Production, v. 221, n. 1, p. 502-508, 2019).

### Pulsed Electric Field (PEF) Technique:

Permeabilization of cell walls of various biological systems can be achieved using pulsed electric fields (PEF). The operating principle of the PEF treatment technique is based on applying short electrical pulses (from a few nanoseconds to a few milliseconds) of high voltage (0.1 to 80 kV cm-1) to the product between two electrodes. A critical transmembrane potential is generated by discharging high-intensity electric field pulses to promote membrane permeabilization (electroporation). This technique can change cell characteristics due to the appearance of pores on the membrane, increasing their permeability and promoting cytoplasm solubilization. A basic PEF device typically consists of a pulse generator, a treatment chamber, and electrodes, with the electrical pulse placed between or on two electrodes (Wang, M. et al. Pulsed electric field (PEF) recovery of biomolecules from Chlorella: Extract efficiency, nutrient relative value, and algae morphology analysis. Food Chemistry, v. 404, p. 134615, 2023; Zhou, L. et al. Bioactive compounds in microalgae and their potential health benefits. Food Bioscience, p. 101932, 2022; Naliyadhara, N. et al. Pulsed electric field (PEF): Avant-garde extraction escalation technology in food industry. Trends in Food Science & Technology, v. 122, p. 238-255, 2022; Gao, W. et al. Polysaccharides in selenium-enriched tea: Extraction performance under innovative technologies and antioxidant activities. Foods, v. 11, n. 17, 2545, 2022; Bensalem, S. et al. Impact of pulsed electric fields and mechanical compressions on the permeability and structure of Chlamydomonas reinhardtii cells. Scientific Reports, v. 10, n. 1, p. 1-11, 2020; Gateau, H. et al. Application of pulsed electric fields for the biocompatible extraction of proteins from the microalga Haematococcus pluvialis. Bioelectrochemistry, v. 137, p. 107588, 2021; Carullo, D. et al. Application of pulsed electric fields and high-pressure homogenization in biorefinery cascade of C. vulgaris microalgae. Foods, v. 11, n. 3, p. 1-16, 2022).

### Acid Treatment Technique:

Cell disruption by acid can be performed using organic acids, inorganic acids, and ionic liquids. Organic acids are less toxic and more biodegradable than ionic liquids and inorganic acids. However, inorganic acids are effective in disrupting microalgae cell walls and can act as catalysts, allowing for higher lipid, carbohydrate, and protein yields when used at low temperatures and short reaction times. Temperatures ranging from 110° C. to 150° C. and acid concentrations from 1% to 1.5% (w v-1) are suitable operating conditions for acid cell wall disruption (Lee, I.; Han, J-I. Hydrothermal-acid treatment for effectual extraction of eicosapentaenoic acid (EPA)-abundant lipids from Nannochloropsis salina. Bioresource Technology, v. 191, p. 1-6, 2015; Nitsos, C. et al. Current and novel approaches to downstream processing of microalgae: A review. Biotechnology Advances, v. 45, 107650, 2020; Rizza, L. S. et al. Bioprospecting for native microalgae as an alternative source of sugars for the production of bioethanol. Algal Research, v. 22, p. 140-147, 2017; Vechio, H. et al. A new approach on astaxanthin extraction via acid hydrolysis of wet Haematococcus pluvialis biomass. Journal of Applied Phycology, v. 33, n. 5, p. 2957-2966, 2021).

### Surfactant Disruption Technique:

Surfactants interact with phospholipids in the cell wall, promoting cell rupture and simultaneously allowing the release of biomolecules. Surfactants are an economically and environmentally viable alternative for microalgae cell disruption when raw material harvesting conditions are unfavorable (Huang, W.; Kim, J. Cationic surfactant-based method for simultaneous harvesting and cell disruption of microalgal biomass. Bioresource Technology, v. 149, p. 579-581, 2013; Lai, Y. S. et al. Cell disruption by cationic surfactants affects bioproduct recovery from Synechocystis sp. PCC 6803. Algal Research, v. 34, p. 250-255, 2018; Taghavijeloudar, M. et al. Simultaneous harvesting and extracellular polymeric substances extrusion of microalgae using surfactant: Promoting surfactant-assisted flocculation through pH adjustment. Bioresource Technology, v. 319, e124224, 2021). However, this method has the disadvantage of contaminating the product, leading to high costs for downstream processing.

### Enzymatic Technique:

Enzymatic hydrolysis is a non-mechanical disruption technique with the advantage of the sustainability of biological enzymes for cell disruption. However, the hydrolysis efficiency with individual enzymes is low, requiring the combined use of multiple enzymes. This significantly increases process costs and is not yet suitable for industrial scale development (Zhang, Y. et al. Assessment of enzyme addition strategies on the enhancement of lipid yield from microalgae. Biochemical Engineering Journal, 177, e108198, 2022; Zhang, J. et al. Utilization of enzymatic cell disruption hydrolysate of Chlorella pyrenoidosa as potential carbon source in algae mixotrophic cultivation. Algal Research, v. 45, e101730, 2020; Zhang, Y. et al. Assessment of enzyme addition strategies on the enhancement of lipid yield from microalgae. Biochemical Engineering Journal, 177, e108198, 2022; Sierra, L. S. et al. Enzymatic cell disruption of the microalgae Chlamydomonas reinhardtii for lipid and protein extraction. Algal Research, v. 25, p. 149-159, 2017; Souza, M. F. et al. Effect of milling and enzymatic hydrolysis in the production of glucose from starch-rich Chlorella sorokiniana biomass. Algal Research, v. 50, p. e101961, 2020).

Mechanical disruption methods are most frequently used because they completely destroy cell walls and are not dependent on the specific chemical composition of microalgae cell walls. However, mechanical cell disruption requires high energy consumption and expensive equipment. In contrast, non-mechanical methods, such as chemical and enzymatic techniques, do not require large equipment investment but depend on cell wall characteristics and are prone to product contamination by exogenous chemicals/enzymes or decomposition of some components in the presence of exogenous chemicals/enzymes. Therefore, the choice of cell disruption technique will primarily depend on the structural and morphological characteristics of the microalgae, the applicability and scalability of the process, and the energy/production cost consumption. It can be said that, to date, cell disruption related to energy efficiency and selective extraction of bioactive compounds from microalgae largely remains unexplored.

On the other hand, lipid separation from microalgae depends not only on the microalgae cell disruption method but also on many other factors, such as microalgae species and extraction method. For example, the total lipid content for Schizochytrium sp. is approximately 50% to 70% of dry biomass; for Nannochloropsis sp., it is approximately 30% to 50% of dry biomass; for Chlorella sp., it is approximately 15% to 40% of dry biomass (Islam, Md. Mirazul & Dixit, Shubha. An Overview Study of Lipid Extraction Methods from Microalgae, SSRN Electronic Journal, 2024; A. Sanchez-Bayo et al. Biodiesel and biogas production from Isochrysis galbana using dry and wet lipid extraction: a biorefinery approach. Renew. Energy, v 146, p. 188-195, 2020; Alavijeh R. S. et al. Combined bead milling and enzymatic hydrolysis for efficient fractionation of lipids proteins, and carbohydrates of Chlorella vulgaris microalgae. Bioresource Technology, v. 309, p. 123321, 2020; Corrêa P S et al. Microalgae Biomolecules: Extraction, Separation and Purification Methods. Processes, v. 9, p. 10, 2021; Georgiopoulou I et al. Process Optimization of Microwave-Assisted Extraction of Chlorophyll, Carotenoid and Phenolic Compounds from Chlorella vulgaris and Comparison with Conventional and Supercritical Fluid Extraction. Applied Sciences, v. 13, p. 2740, 2023; González-González et al. Osmotic shock pre-treatment of Chaetoceros muelleri wet biomass enhanced solvent-free lipid extraction and biogas production. Algal Research, v. 54, p. 102177, 2021; González-González et al. Impact of osmotic shock pre-treatment on microalgae lipid extraction and subsequent methane production. Bioresour. Technol. Rep., v. 7, p. 10021, 2019; He, Y. Z. et al. Sustainable biodiesel production from the green microalgae Nannochloropsis: Novel integrated processes from cultivation to enzyme-assisted extraction and ethanolysis of lipids. Energy Conversion and Management, v. 209, p. 112618, 2020; Jesus, S. D. et al. Comparison of several methods for effective lipid extraction from wet microalgae using green solvents. Renewable Energy, v. 143, p. 130-141, 2019; K. K. Asanka Sanjeewa et al. Enzyme-assisted extraction of bioactive compounds from seaweeds and microalgae. TrAC Trends in Analytical Chemistry, v. 167, p. 117266, 2023; Koyande, A. et al. Integration of osmotic shock assisted liquid biphasic system for protein extraction from microalgae Chlorella vulgaris. Biochem. Eng. J., v. 157, p. 107532, 2020; Kumari, N. et al. Biofuel and co-products from algae solvent extraction. Journal of Environmental Management, v. 247, p. 196-204, 2019; M. Kwak, S. K. et al. Simultaneous cell disruption and lipid extraction of wet Aurantiochytrium sp. KRS101 using a high shear mixer. Bioprocess Biosyst. Eng., p. 1-8, 2018; R. Kapoore, T. B. Microwave-assisted extraction for microalgae: from biofuels to biorefinery. Biology (Basel), v. 7, p. 18, 2018; Rajauria, G. Enzyme-assisted extraction of bioactives from plants. Trends in Biotechnology, 36 (4), 388-399, 2018; Wang T, Z. L. Extraction and Separation of Natural Products from Microalgae and Other Natural Sources Using Liquefied Dimethyl Ether, a Green Solvent: A Review. Foods, v. 13, p. 352, 2024; Yao, S. M. The effect of high-intensity ultrasound on cell disruption and lipid extraction from high-solids viscous slurries of Nannochloropsis sp. biomass. Algal Research, v. 35, p. 341-348, 2018; Zhang, Y. K. Optimization of enzymatic hydrolysis for effective lipid extraction from microalgae Scenedesmus sp. Renewable Energy, 125, 1049-1057, 2018; J. W. Extraction of lipids from microalgae using classical and innovative approaches. Food Chemistry, v. 371, p. 131141, 2022).

Methods for extracting total lipids from microalgae biomass include hot water extraction under subcritical water conditions (Subcritical Water Extraction, SWE) and solvent extraction. The SWE method uses water at temperatures ranging from 120° C. to 200° C. and pressures from 10 bar to 20 bar to disrupt cells and release lipids, yielding a total lipid extraction efficiency ranging from 60% to 80%. The advantage of this method is its safety and the absence of organic solvents. However, the disadvantage is that under subcritical temperature and pressure conditions, water can act as an oxidizing agent, leading to oxidation of lipid products and thus a decrease in oil quality, DHA/EPA. The most common method today is solvent extraction. Specifically, the Bligh & Dyer method, which uses a chloroform/methanol solvent to extract total lipids from dry biomass, achieves a total lipid extraction efficiency ranging from 85% to 95%. The principle of this method is to dissolve lipids in a chloroform/methanol mixture (2:1) to achieve maximum lipid recovery. The disadvantage of this method is the use of toxic solvents, which are not suitable for food applications. Moreover, this method is only suitable for laboratory use and is considered an experimental method for determining total lipid content in microalgae samples. Soxhlet extraction with hot organic solvents (hexane, ethanol/isopropanol) to extract total lipids from dry biomass yields an extraction efficiency ranging from 80% to 90%. The disadvantage of this method is that it requires a long time and consumes a lot of solvent. Supercritical CO₂ extraction, used to extract total lipids from dry biomass, uses CO₂ in a supercritical state as a solvent, achieving an extraction efficiency ranging from 70% to 85%. The advantage of this method is that it does not use chemical solvents, making it safe for food and pharmaceutical applications. However, the disadvantage is that the process operates at high pressure (~300 bar), requiring expensive equipment and making scale-up difficult.

Another lipid separation method of interest is cold pressing. The advantage of cold pressing is that it preserves oil quality (does not denature DHA, EPA, and other bioactive compounds); it does not require chemical solvents, making it safer for food and pharmaceutical applications; and it maintains the natural flavor and color of the oil (preserving the original properties of microalgae oil). The raw material for cold pressing is typically dry microalgae biomass, dried by freeze-drying or vacuum drying, to a post-drying moisture content of less than 5% to prevent raw material oxidation and reduce pressing efficiency. Using fresh microalgae (over 80% moisture) yields very low pressing efficiency because oil is retained in water. This process typically uses screw presses or membrane presses with the following conditions:

Pressing temperature: ≤50° C. (preferably ranging from 35° C. to 45° C.).

Pressing pressure: ranging from 30 MPa to 100 MPa (depending on the technology).

Pressing speed: slow pressing to limit heat generation.

Pressing environment: use oxygen-free gas to reduce oil oxidation.

After cold pressing, microalgae oil may contain algae residue or small impurities, so further purification may be required by:
Coarse filtration: Through a membrane filter with pore sizes ranging from 1 micron to 5 microns to remove algae cake.
High-speed centrifugation: Separating pure oil from water and impurities.
Nanofiltration (Membrane Filtration): Retaining DHA and removing small impurities.

The biggest disadvantage of the cold pressing method is its very low oil separation efficiency, only reaching 15% to 50% (depending on the algae species), which is much lower than the methods mentioned above (solvent extraction or supercritical CO₂ extraction).

On the other hand, lipid extraction methods by cold pressing or by using solvents or supercritical CO₂ first require drying the microalgae biomass, which hinders economic feasibility due to high energy demand and/or the need for expensive equipment investment for the drying process. Furthermore, the drying process (if high temperatures are used) can more or less affect the quality of microalgae lipids due to thermal denaturation of heat-sensitive components.

Therefore, there is an urgent need to find a high-efficiency lipid extraction process directly from wet biomass, under mild conditions (low temperature, atmospheric pressure), capable of industrial-scale production, yielding high-quality pure microalgae oil, to achieve significant economic-technical and environmental efficiency, addressing the problems mentioned above.

However, to date, no published work has disclosed a highly efficient lipid separation process directly from wet microalgae biomass at low temperature and atmospheric pressure.

Based on the above, it is recognized that finding a highly effective cell wall disruption process for wet microalgae biomass combined with a process for separating lipids and valuable compounds that does not denature DHA, EPA, and other bioactive compounds, and can be implemented at an industrial scale, is an urgent need. This invention was created on that basis.

### SUMMARY

The purpose of the invention is to propose a process of producing microalgae oil that is highly efficient, consumes less energy, is environmentally friendly, and is capable of industrial-scale production, yielding high-quality pure microalgae oil, bringing significant economic-technical and environmental benefits.

To achieve the above purpose, in one aspect, the invention proposes a microalgae cell disruption process comprising pre-milling wet microalgae biomass using a milling bead to preliminarily disrupt cell membranes, then performing a first sonication of the ground microalgae biomass to weaken cell walls; mixing the sonicated microalgae biomass to activate intracellular enzymes; and finally, a second sonication of the wet biomass to break down remaining cell walls, helping to enhance oil recovery in subsequent steps.

The sonication and mixing processes are performed in a single device that has both stirring blades for mixing microalgae biomass and integrated ultrasonic probes. The combination of bead milling for preliminary cell disruption with the use of a mixing device equipped with ultrasonic probes for cell disruption significantly reduces the time and energy required for microalgae cell disruption, thereby reducing heat generation and thus preserving product quality, avoiding denaturation of heat-sensitive products, and increasing the recovery efficiency of microalgae oil and valuable active ingredients from microalgae biomass.

In another aspect, the invention proposes a process for producing microalgae oil from wet microalgae biomass, including steps of: bead milling for preliminary cell disruption; first sonication, mixing, and second sonication in a mixing device equipped with ultrasonic probes to disrupt microalgae cell walls by physical action, combined with autolysis of cell walls by intracellular enzymes; followed by separating oil and valuable active ingredients from microalgae biomass by primary and secondary centrifugation; then centrifuging in a conical centrifuge to obtain microalgae oil and a liquid phase containing valuable water-soluble active ingredients, and finally concentrating this liquid phase to obtain valuable water-soluble products.

In yet another aspect, the invention proposes microalgae oil and valuable products obtained by the production process described above.

More specifically, the invention proposes a process of producing microalgae oil from wet microalgae biomass comprising the following steps:

Step 1: Load wet microalgae biomass into a bead mill and pre-mill the wet microalgae.

Step 2: Treat to activate intracellular enzymes to disrupt cell walls and microalgae cells.

Transfer the pre-milled microalgae biomass obtained in Step 1 to a mixing device equipped with ultrasonic probes in an inert gas atmosphere. Perform a first sonication until the entire microalgae biomass reaches a temperature ranging from 30° C. to 55° C. Stop sonication, maintain stable biomass temperature, and mix the microalgae biomass for sufficient time for the intracellular enzymes present in the microalgae biomass to break down microalgae cell walls, facilitating the release of microalgae oil from the cells. Then, stop mixing and perform a second sonication under similar conditions as the first sonication, to enhance the degree of microalgae cell wall disruption, thereby increasing oil separation efficiency.

Step 3: Perform primary centrifugation to separate the microalgae biomass phase obtained in Step 2 to separate the liquid phase containing most of the crude oil (still mixed with water) and valuable water-soluble active ingredients from the microalgae biomass cake. The liquid phase obtained in this step is called primary crude oil, and the sludge obtained in this step is called primary sludge.

Step 4: Centrifuge the primary crude oil obtained in Step 3 in a conical centrifuge to separate the oil phase from the water phase, where the water phase (also called primary liquid) contains water and valuable water-soluble active ingredients, and the oil phase contains microalgae oil.

Step 5: Return the primary sludge obtained in Step 3 and the primary liquid obtained in Step 4 to the mixing device equipped with ultrasonic probes to perform sonication on the mixture comprising primary sludge and primary liquid in an inert gas atmosphere.

Step 6: Perform secondary centrifugation of the mixture comprising primary sludge and primary liquid that has been sonicated in Step 5 to separate the oil phase (still mixed with water) from the solid residue. The oil phase (still mixed with water) obtained is called secondary crude oil, and the solid residue obtained in this step is called secondary sludge.

Step 7: Perform centrifugation of the secondary crude oil obtained in Step 6 in a conical centrifuge to separate the oil phase from the water phase to obtain microalgae oil and a water phase containing water and valuable water-soluble active ingredients, this water phase is called secondary liquid.

Step 8: Vacuum filter the secondary sludge obtained in Step 6 to separate the liquid, called vacuum liquid, and obtain microalgae residue. The secondary liquid and vacuum liquid are mixed together and vacuum concentrated to obtain a concentrated liquid product containing valuable components.

The microalgae oil product obtained according to the invention is pure oil, not refined, with high value due to mild oil separation conditions, ensuring no denaturation of DHA, EPA, and other bioactive compounds, while preserving the original properties of microalgae oil.

### BRIEF DESCRIPTION OF THE DRAWING

FIG. 1 illustrates a process of producing microalgae oil from wet microalgae biomass according to the invention.

### DETAILED DESCRIPTION

The term “wet microalgae biomass” as used in this invention means microalgae biomass obtained from cultivation, after having been passed through a centrifuge to separate the biomass from the culture medium. Wet microalgae biomass typically contains 75% to 90% water, depending on the microalgae species and harvesting method.

The aforementioned wet microalgae raw material includes at least one of the oil-producing microalgae species selected from Nannochloropsis sp., Chlorella sp., Dunaliella sp., Isochrysis sp., Phaeodactylum tricornutum sp., Botryococcus braunii sp., Scenedesmus sp., Schizochytrium sp., Crypthecodinium sp. obtained from microalgae harvesting, without the need for a drying process.

The term “intracellular enzymes” as used in this invention means enzymes synthesized and active inside microalgae cells to maintain essential biochemical processes for life, which are activated after microalgae harvesting to support the autolysis of microalgae cell walls during microalgae processing for oil separation.

The term “milling bead ratio” as used in this invention means the mass ratio between beads and the material being milled, expressed as bead mass/material (kg/kg).

The term “exogenous enzymes” as used in this invention means enzymes introduced from an external source.

The concept of “cell wall autolysis by intracellular enzymes” as used in this invention means the process in which cell walls self-decompose due to activated intracellular enzymes, helping to release lipids, proteins, and carbohydrates without mechanical or chemical intervention.

The term “synergistic effect” as used in this invention means the effect obtained by combining multiple mechanical, chemical, or biological methods that can significantly improve cell wall disruption efficiency compared to each method individually, thereby substantially increasing the efficiency of intracellular compound release.

The combination of processes, including preliminary bead milling, short-duration sonication, and mixing to activate intracellular enzymes, helps to leverage the advantages of each method while limiting their disadvantages: preliminary bead milling will help to initially rupture cell membranes, especially for microalgae species with thick and rigid cell walls such as Chlorella sp., increasing the contact surface with microbubbles generated by ultrasonic waves, thereby allowing ultrasonic waves to burst or weaken the already ruptured cell walls. Simultaneously, the mixing process at appropriate temperatures and durations activates intracellular enzymes within microalgae cells, enabling these enzymes to hydrolyze cell walls more efficiently. The combination of methods significantly reduces the time and energy required to disrupt microalgae cell walls, thereby reducing heat generation and thus preserving product quality, avoiding denaturation of heat-sensitive products, and increasing the efficiency of lipid and valuable active ingredient extraction from microalgae.

The concept of “primary and secondary centrifugation” in this invention means the centrifugation process to separate oil and valuable active ingredients from wet microalgae biomass, repeated twice. In the first oil separation, also called primary centrifugation, wet microalgae biomass that has undergone bead milling for cell disruption is introduced into a phase separation centrifuge to separate crude microalgae oil mixed with water from the microalgae biomass, yielding primary crude oil and primary sludge. Then, the entire primary sludge is returned to the mixing and sonication device for another sonication, and then introduced into the phase separation centrifuge to perform the second oil separation (secondary centrifugation). Primary and secondary centrifugation use a phase separation centrifuge, which is considered one of the effective devices for separating oil from biomass sludge. A phase separation centrifuge is a horizontal centrifuge that uses centrifugal force to separate components in a mixture with different densities. Under the action of centrifugal force, components with high density (such as biomass sludge) will move outwards, while lighter components (such as crude oil) will accumulate in the center. The biomass sludge will be compressed and discharged, while crude microalgae oil mixed with water will flow out as a liquid.

The term “primary crude oil” in this invention means oil mixed with water obtained from the first oil separation from wet microalgae biomass in a phase separation centrifuge.

The term “secondary crude oil” in this invention means oil mixed with water obtained from the second oil separation from wet microalgae biomass that has undergone the first oil separation, in a phase separation centrifuge.

The concept of “crude microalgae oil mixed with water” in this invention means the liquid separated from wet microalgae biomass, whose composition is oil mixed with water and valuable water-soluble active ingredients. Usually, the oil phase and water phase are immiscible and typically separate into two phases. However, for crude microalgae oil mixed with water separated from microalgae biomass, water and valuable water-soluble active ingredients disperse into the oil phase as emulsion particles. Generally, according to the process in this invention, the process of separating oil and valuable active ingredients from wet microalgae biomass is performed twice, so crude microalgae oil mixed with water is a mixture of primary crude oil (obtained from the first oil separation from wet microalgae biomass) and secondary crude oil (obtained from the second oil separation from wet microalgae biomass that has undergone the first oil separation). After obtaining crude microalgae oil mixed with water, it is necessary to separate water and valuable water-soluble active ingredients from the oil phase using a conical centrifuge to obtain pure microalgae oil.

The term “pure microalgae oil” in this invention means high-quality, unrefined microalgae oil, directly separated from wet microalgae biomass without any refining process, due to the extraction process being performed at low temperatures, in an inert gas atmosphere, without the addition of any chemicals or extraction solvents, including exogenous enzymes. This microalgae oil separation method ensures the purity of the oil and does not denature valuable nutritional components in microalgae oil such as omega-3 (DHA, EPA), while retaining valuable active ingredients that are soluble or partially soluble in microalgae oil (including carotenoids—powerful antioxidants such as lutein, zeaxanthin, β-carotene, astaxanthin, etc., and chlorophyll, a substance that helps detoxify the body and supports hemoglobin production) by avoiding oxidation that denatures the product. Pure microalgae oil is the highest grade of microalgae oil.

The term “valuable active ingredients” in this invention means water-soluble active ingredients separated from microalgae cells along with crude microalgae oil. These valuable active ingredients include, but are not limited to, protein and peptide compounds (containing up to 20 types of amino acids, including branched-chain amino acids (BCAA) such as Leucine, Isoleucine, Valine, which are essential amino acids that the body cannot synthesize and must be supplemented through food or dietary supplements); Chlorella Growth Factor (CGF), in cases where the raw material is Chlorella sp. microalgae, containing nucleotides, peptides, polysaccharides, vitamins, and minerals, which promote cell growth, boost immunity, and support digestion; Polysaccharides (long-chain sugars, including β-glucans, rhamnan sulfates, arabinose, glucose, which enhance the immune system, anti-inflammatory, stimulate gut microbiota; B vitamins (B1, B2, B6, B12) and Vitamin C, which boost energy, support metabolism, and are good for the nervous system; Minerals (Fe, Mg, Zn, K, Ca, P, Se . . . ), essential for growth, metabolism, and enzyme function; Nucleic acids (DNA, RNA), which help regenerate cells, anti-aging, and support immune function.

The term “valuable product” in this invention means a product containing valuable active ingredients, which have been separated from microalgae cells along with crude microalgae oil and then separated from the oil in the form of a liquid, consisting of water and valuable water-soluble active ingredients, subsequently concentrated under vacuum at low temperature.

The term “primary sludge” as used in this invention means the residue of wet microalgae biomass after primary crude oil has been separated (corresponding to the first oil separation). Typically, microalgae oil cannot be completely separated from microalgae biomass with just one separation, so primary sludge still contains a certain amount of oil. Further oil separation from primary sludge by a second separation will contribute to increasing the total efficiency of microalgae oil recovery from wet biomass.

The term “secondary sludge” as used in this invention means the residue of wet microalgae biomass after secondary crude oil has been separated (corresponding to the second oil separation).

The term “microalgae residue” as used in this invention means the solid residue obtained after subjecting secondary sludge to vacuum filtration to separate the liquid, consisting of water and valuable water-soluble active ingredients. Microalgae residue, after separation of oil and valuable active ingredients, still contains many useful nutrients such as proteins, carbohydrates, minerals (N, P, K), and other trace elements (Fe, Mg, Ca, Zn), beneficial microorganisms, antioxidants, and cellulose. Microalgae residue is utilized as a good raw material for producing organic fertilizers, animal feed preparations, biochar, raw materials for the pharmaceutical and cosmetic industries (antioxidants, skin cleansers, skin care products), biofuels such as biogas and bioethanol, raw materials in the production of functional foods, and the production of biomaterials and composite materials.

The term “phase separation centrifuge” in this invention means a phase separation centrifuge capable of separating the oil-water phase from the sludge to obtain crude microalgae oil mixed with water and sludge.

The term “solid content” in this invention means the content of microalgae residue (dry basis) in the liquid fed into the phase separation centrifuge.

The term “conical centrifuge” in this invention means a device used to separate the water phase from crude microalgae oil mixed with water to obtain pure microalgae oil product and a liquid phase, consisting of water and valuable water-soluble active ingredients. A conical centrifuge is a high-speed centrifuge used to separate liquid-liquid phases by strong centrifugal force. The device has a special structure with multiple conical discs stacked on top of each other to increase separation efficiency. As a result, a conical centrifuge can thoroughly separate the water phase from the oil, producing pure microalgae oil product with a water content of 0.1%. In addition, the device has high separation efficiency, capable of fast, continuous processing, reducing raw material loss.

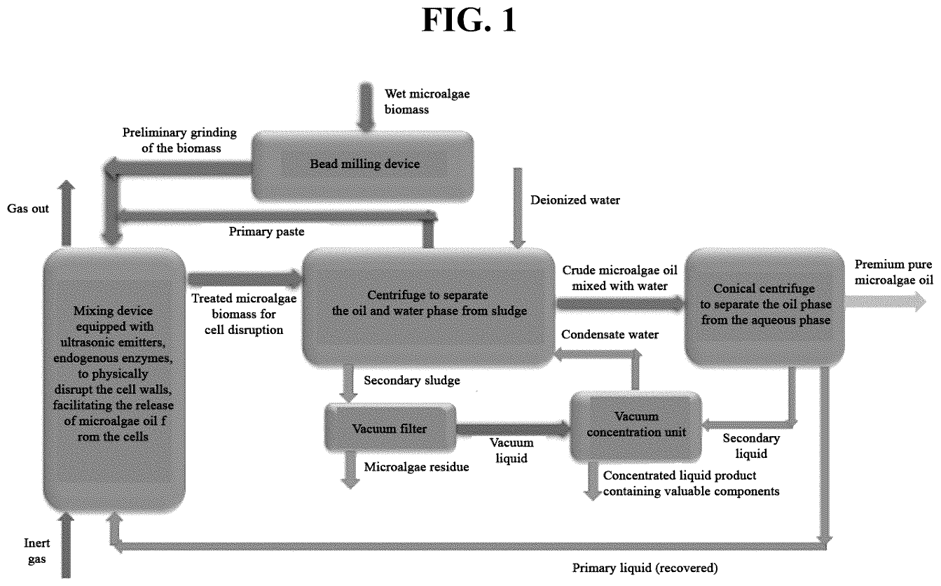

The microalgae oil production process according to the invention is illustrated in FIG. 1. Specifically, the process according to the invention includes the following steps:
Load wet microalgae biomass into a bead mill, with zirconia silicate milling beads with a diameter ranging from 0.5 mm to 3.0 mm, a bead to biomass ratio of 1/1 to 2/1, and pre-mill the wet microalgae for 5 minutes to 20 minutes, with a milling chamber rotation speed ranging from 1000 rpm to 3000 rpm.
Next, the pre-milled microalgae are separated from the milling beads and transferred to a mixing device equipped with ultrasonic probes, such that the ultrasonic probes are submerged in the microalgae biomass; close the lid of the device, feed inert gas to purge air from the device, creating an inert gas atmosphere in the device; perform a first sonication at a frequency ranging from 20 kHz to 40 kHz, power ranging from 2000 W to 4000 W until the microalgae biomass reaches a temperature ranging from 30° C. to 55° C.; stop sonication; turn on heating to maintain the biomass temperature stable, at a temperature ranging from 30° C. to 55° C.; turn on the stirring blades to perform biomass mixing; mix the biomass for a period of 20 minutes to 50 minutes, sufficient for the intracellular enzymes present in the microalgae biomass to break down microalgae cell walls, facilitating the release of microalgae oil from the cells; stop mixing, perform a second sonication at a frequency ranging from 20 kHz to 40 kHz, power ranging from 2000 W to 4000 W, for a period of 2 minutes to 8 minutes, to enhance the degree of microalgae cell wall disruption, thereby increasing oil separation efficiency.
The microalgae biomass that has undergone the above pre-treatment steps is transferred to a phase separation centrifuge; operate the device at a centrifugation speed ranging from 4000 rpm to 6000 rpm, to perform the step of separating most of the microalgae oil from the microalgae biomass sludge. The oil obtained at this stage is called primary oil. The sludge after primary oil separation is called primary sludge.
The primary crude oil exiting the phase separation centrifuge is transferred to a conical centrifuge to separate the water phase, including water and valuable water-soluble active ingredients, from the oil phase, to obtain pure microalgae oil and primary liquid, consisting of water and valuable water-soluble active ingredients. The speed of the conical centrifuge ranges from 5000 rpm to 7000 rpm.
Primary sludge is returned to the mixing device equipped with ultrasonic probes. The primary liquid, consisting of water and valuable water-soluble active ingredients, separated from the oil in the conical centrifuge mentioned above, is also recirculated back to the mixing device equipped with ultrasonic probes, to be mixed with the primary sludge. Feed inert gas into the mixing device equipped with ultrasonic probes to purge air from the device, creating an inert gas atmosphere in the device. Perform sonication on the mixture comprising primary sludge and primary liquid, at a frequency ranging from 20 kHz to 40 kHz, power ranging from 2000 W to 4000 W, for a period of 2 to 8 minutes.
Then, pump the mixture comprising primary sludge and primary liquid that has undergone sonication to a phase separation centrifuge to perform the secondary oil separation step, separating most of the oil and valuable water-soluble active ingredients, remaining from the primary separation, from the sludge. The crude oil (still mixed with water) obtained at this stage is called secondary crude oil. The sludge obtained at this stage is called secondary sludge.
Transfer the secondary crude oil to a conical centrifuge to separate the water phase containing water and valuable water-soluble active ingredients from the oil phase to obtain pure microalgae oil and a liquid phase containing water and valuable water-soluble active ingredients, which is called secondary liquid.
The secondary sludge is transferred to a vacuum filter to separate the solid residue from the liquid, which is called vacuum liquid, and the solid residue obtained is used as raw material for several processing steps in agriculture, food, and industry.
The secondary liquid and vacuum liquid are mixed together and vacuum concentrated to the desired concentration, yielding a concentrated liquid product containing valuable components.

According to the invention, wet microalgae biomass (water content of about 75% to 90%) is pre-milled in a bead mill using zirconia silicate milling beads with a diameter ranging from 0.5 mm to 3.0 mm, more preferably from 0.5 mm to 2.5 mm, most preferably from 1.0 mm to 2.0 mm. In general, the size of milling beads strongly affects the efficiency of microalgae biomass milling. Small beads (diameter of less than 0.5 mm) create high shear force, suitable for breaking thin-walled microalgae cell walls and achieving high fineness, suitable for lipid or protein extraction, but are prone to clumping, difficult to mix in wet milling systems, and yield lower milling efficiency due to low impact energy. Medium beads (diameter of from 0.5 mm to 2.0 mm) are the optimal choice for most microalgae, thanks to a balance between cell disruption rate and milling efficiency, while reducing heat generation compared to small beads, helping to protect bioactive compounds such as unsaturated fatty acids (DHA, EPA), proteins, carotenoids. Large beads (diameter of over 2 mm) are suitable for microalgae with thick walls due to strong impact force, providing faster milling speed, reducing milling time, but are prone to heat generation, which can affect the quality of oil or protein in microalgae, and produce a less fine milled product.

The milling beads used in the invention are made of zirconia silicate because glass milling beads are suitable for gentle milling, which does not denature bioactive compounds, but are not suitable for microalgae with thick and rigid cell walls, while stainless steel milling beads can cause high heat generation, which is not suitable for heat-sensitive compounds. Zirconia silicate milling beads have suitable hardness for disrupting thick and rigid microalgae cell walls, while generating less heat during milling.

Preferably, if the bead to biomass ratio ranges from 5/1 to 30/1, more preferably from 10/1 to 25/1, most preferably from 15/1 to 25/1. A low bead to biomass ratio below 5/1 will result in less impact, reduced friction, and thus less heat generation, preserving heat-sensitive bioactive compounds (DHA, EPA, proteins, carotenoids), but with low milling efficiency, incomplete cell disruption, and large particle size of the milled product, making lipid or protein extraction difficult. A high bead to biomass ratio above 25/1 creates strong impact and shear force, faster milling, and very fine particle size, suitable for lipid or protein extraction, but generates high heat, which can inactivate the biological activity of oil, protein, and enzymes, and increases milling bead wear and operating energy consumption. A bead to biomass ratio ranging from 15/1 to 25/1 creates a balance between beads and raw material, thereby creating a balance between cell disruption rate and energy consumption. Such a bead to biomass ratio is suitable for microalgae with medium to thick and rigid cell walls, such as Nannochloropsis sp., Chlorella sp., Botryococcus sp., and also reduces the loss of bioactive compounds, maintaining the quality of microalgae oil.

Preferably, the preliminary milling time ranges from 30 minutes to 4 hours, more preferably from 1 hour to 3 hours, most preferably from 1 hour 30 minutes to 2 hours. A too short milling time (under 30 minutes) will save energy and generate less heat, but the cell disruption efficiency for microalgae species with thick and rigid cell walls is low. A too long milling time (over 4 hours) will ensure the highest fineness, but will generate high heat, which can damage lipids (DHA, EPA), proteins, and consume a lot of energy. Furthermore, milling efficiency will not increase significantly after a certain period. Therefore, for the purpose of preliminary milling to partially disrupting cell walls, a milling time of 30 minutes to 4 hours is suitable, ensuring a balance between milling efficiency and the ability to protect bioactive compounds and is suitable for extracting lipids, proteins, and carotenoids.

For a milling chamber with a volume of 15 liters, preferably the milling chamber rotation speed ranges from 30 rpm to 60 rpm, more preferably from 40 rpm to 60 rpm, most preferably from 40 rpm to 54 rpm.

The step of separating pre-milled microalgae from milling beads is performed by gravity settling, by allowing the system to stand for about 5 minutes to 15 minutes. Milling beads settle to the bottom, and the microalgae suspension above is collected.

The wet microalgae biomass raw material used in this invention is wet microalgae paste, harvested and separated from the culture medium by any water separation method. The wet microalgae biomass is used directly for cell wall disruption and lipid separation without the need for drying or any other processing.

Microalgae raw material can be obtained or sourced from any cultivation process or cultivated specifically to maximize yield and oil content. For example, among the microalgae species cultivated at an industrial scale, Chlorella sorokiniana has been experimentally proven to be a unicellular green microalga with many applications in the food, pharmaceutical, biofuel, and environmental industries, thanks to its autotrophic and heterotrophic living characteristics (can grow in both light and organic nutrient environments), rapid growth (higher growth rate than other Chlorella species), and high lipid content. The cell wall of Chlorella sorokiniana is quite thick, containing cellulose and hemicellulose, making cell disruption more difficult than other microalgae species, so high-efficiency lipid separation for Chlorella sorokiniana is very significant in terms of technology. Other microalgae species that have been cultivated at an industrial scale include Nannochloropsis sp., Chlorella vulgaris, Scenedesmus obliquus, Botryococcus braunii, Dunaliella tertiolecta, Neochloris oleoabundans, Isochrysis galbana, Phaeodactylum tricornutum. Other microalgae genera/species can also be used in this process.

The complete disruption of microalgae cell walls is performed in a mixing device equipped with ultrasonic probes, such that the ultrasonic probes are submerged in the microalgae biomass, approximately 10 cm to 40 cm, more preferably 10 cm to 30 cm, most preferably 10 cm to 20 cm. If the upper surface level of the microalgae biomass is much higher than the position of the ultrasonic probes in the device, the sonication efficiency for the uppermost biomass will be limited. If the upper surface level of the microalgae biomass is very close to the position of the ultrasonic probes in the device, the treatment efficiency is reduced.

After loading the wet biomass, close the lid of the device, feed inert gas to purge air from the device, creating an inert gas atmosphere in the device. The inert gas can be nitrogen or argon. Nitrogen gas is a common, low-cost inert gas that can be produced on-site by a nitrogen generator; while argon gas, although not common, is heavier than air, so in some cases, argon gas is preferred because it is more effective in creating an inert environment than nitrogen gas.

Microalgae biomass after bead milling is subjected to microalgae cell wall disruption in a mixing device equipped with ultrasonic probes, in an inert gas atmosphere. This process is performed by combining sonication using ultrasonic probes with a mixing process using intracellular enzymes at low temperature.

Preferably, sonication is performed at a frequency ranging from 20 kHz to 40 kHz, more preferably from 20 kHz to 35 kHz, most preferably from 20 kHz to 30 kHz.

Preferably, the ultrasonic power ranges from 2000 W to 4000 W, more preferably from 3000 W to 4000 W, most preferably from 3000 W to 4000 W.

Preferably, sonication is performed until the biomass reaches a suitable temperature, ranging from 30° C. to 55° C., more preferably the biomass temperature ranges from 35° C. to 50° C., most preferably the biomass temperature ranges from 40° C. to 45° C.

After sonication, turn on heating to maintain the biomass temperature stable, preferably at a temperature ranging from 35° C. to 55° C., more preferably at a temperature ranging from 35° C. to 50° C., most preferably at a temperature ranging from 40° C. to 45° C. Turn on the stirring blades to perform biomass mixing. Mix the biomass for a suitable period sufficient for the intracellular enzymes present in the microalgae biomass to break down microalgae cell walls, facilitating the release of microalgae oil from the cells.

Preferably, the mixing time ranges from 20 minutes to 50 minutes, more preferably from 25 minutes to 50 minutes, most preferably from 30 minutes to 45 minutes. A too short mixing time is not enough to activate intracellular enzymes to work more strongly, while a too long mixing time does not increase cell disruption efficiency and can also create excessively small water-in-oil emulsion particles, leading to difficulties in subsequent water-oil separation.

After mixing, perform a second sonication at a frequency of 40 kHz, power of 2000 W-4000 W, for a suitable period, to enhance the degree of microalgae cell wall disruption, thereby increasing oil separation efficiency. Preferably, sonication is performed for 1 minute to 15 minutes, more preferably from 1 minute to 10 minutes, most preferably from 2 minutes to 8 minutes.

Then, the microalgae biomass is transferred to a phase separation centrifuge to obtain primary oil and primary sludge. The phase separation centrifugation speed ranges from 3000 rpm to 4000 rpm.

Primary crude oil exiting from the phase separation centrifuge is transferred to a conical centrifuge to separate microalgae oil from the water phase to obtain primary oil product and primary liquid. The speed of the conical centrifuge ranges from 5000 rpm to 7000 rpm.

Primary sludge is returned to the mixing device equipped with ultrasonic probes. The primary liquid separated from the oil in the conical centrifuge is also recirculated back to the mixing device equipped with ultrasonic probes, to be mixed with the primary sludge. Feed inert gas into the mixing device equipped with ultrasonic probes to purge air from the device, creating an inert gas atmosphere in the device.

Perform sonication on the mixture comprising primary sludge and primary liquid, at a frequency ranging from 20 kHz to 40 kHz, power ranging from 2000 W to 4000 W, for a period of 1 minute to 15 minutes, more preferably from 1 minute to 10 minutes, most preferably from 2 minutes to 8 minutes. Then, pump the mixture comprising primary sludge and primary liquid that has undergone sonication to a phase separation centrifuge to perform the step of separating oil from the sludge to separate most of the oil and valuable water-soluble active ingredients remaining from the primary separation, from the primary sludge. The oil (still mixed with water) obtained is called secondary crude oil. Secondary crude oil is transferred to a conical centrifuge to separate the oil phase from the water phase containing water and valuable water-soluble active ingredients to obtain microalgae oil product and a water phase in the form of a liquid containing water and valuable water-soluble active ingredients, which is called secondary liquid.

The sludge after secondary crude oil separation, called secondary sludge, is transferred to a vacuum filter to separate the liquid from the solid residue, this liquid is called vacuum liquid, and the solid residue obtained is used as raw material for several processing steps in agriculture, food, and industry.

The secondary liquid and vacuum liquid are mixed together and vacuum concentrated to the desired concentration, yielding a concentrated liquid product containing valuable components.

According to the invention, the obtained microalgae residue can be used as a raw material for producing organic fertilizers because microalgae residue is rich in nutrients, including essential minerals and trace elements for plants; it can be used as an animal feed preparation because microalgae residue contains many proteins, amino acids, and other nutrients, which are very suitable for supplementing the diet of livestock, poultry, and aquaculture, helping to accelerate growth and improve the health of farmed animals; it can be used to produce biochar; to produce raw materials for the pharmaceutical and cosmetic industries such as antioxidants, skin cleansers, skin care products; to produce biofuels such as biogas and bioethanol; and to be used as raw material in the production of functional foods and composite materials.

The process according to the invention allows for the separation of pure microalgae oil product and valuable water-soluble active ingredients without denaturing the products, thanks to the cell wall disruption process being carried out at low temperatures, facilitating the thorough release of both oleophilic components (oil droplets) and hydrophilic components (water-soluble active ingredients) from the microalgae cells and cell walls. The process is performed under suitable conditions to ensure that the water-in-oil emulsion particles are of appropriate size, not too small, so that the separation of water and water-soluble active ingredients from the oil proceeds smoothly.

In addition to pure microalgae oil products and valuable water-soluble active ingredients with high nutritional value and high added value, the process of the invention also yields solid residue that still contains many valuable active ingredients and is not denatured, thus also being a good raw material for producing a wide range of products in agriculture, industry, food, pharmaceutical, and energy sectors. On the other hand, according to the invention, the process of separating oil and water-soluble compounds from microalgae cells is performed twice, which allows for thorough separation, so the separation efficiency of oil and valuable active ingredients can reach up to 96%. This significantly increases the economic-technical efficiency of the process.

The process according to the invention allows to separate both pure microalgae oil products and valuable water-soluble active ingredients without degrading the products, thanks to the process of breaking the cell wall at low temperature conditions, creating conditions to completely release both the lipophilic components (oil particles) and the hydrophilic components (water-soluble active ingredients) from the cells and cell walls of microalgae. The process is carried out under suitable conditions to ensure that the water-in-oil emulsion particles are of suitable size, not too small, so the process of separating water and water-soluble active ingredients from the oil takes place smoothly.

In addition to pure microalgae oil products and valuable water-soluble active ingredients with high nutritional value and creating high added value, according to the process of the invention, solid residue is also obtained that still contains many valuable active ingredients and is not degenerated, so it is also a good source of raw materials for mass production of products in the fields of agriculture, industry, food and pharmaceuticals, and energy.

On the other hand, according to the invention, the process of separating oil and water-soluble compounds from microalgae cells is repeated through two separations, allowing the separation process to be thorough, so the separation efficiency of oil and valuable active ingredients can be up to 96%. This significantly increases the economic and technical efficiency of the process.

The process according to the invention consumes little energy, first of all thanks to the direct use of wet microalgae biomass, without the need to spend energy on drying, secondly, thanks to the combination of wet bead milling, with a milling time that is shorter than that of current bead milling processes, to preliminarily break the microalgae cell wall, with a short-term ultrasonic process, combined with the self-decomposition of the cell wall by intracellular enzymes that takes place during the mixing process at moderate temperatures. This combination creates a synergistic effect that facilitates the complete release of oil particles and valuable active ingredients from the microalgae cells and cell walls in the next stage, while significantly reducing the time and energy required to break down the microalgae cell walls, resulting in reduced operating costs.

Another advantage of the process according to the invention is that the separation of oil and water-soluble compounds from microalgae cells does not require solvents, chemicals, or exogenous enzymes. Therefore, the final product is a pure extract and does not require further refining and/or complex and costly treatment steps.

### EXAMPLES

The invention will be better understood through the illustrative examples below. These examples are for demonstration purposes only and do not limit the scope of the invention.

### Example 1—Production of Microalgae Oil and Value-Added Products

### Step 1:

Load wet Chlorella sorokiniana biomass (80% moisture content) into a bead milling device. Use zirconium silicate beads with diameters ranging from 1.7 mm to 2.0 mm at a bead-to-biomass ratio of 2:1. Perform a preliminary milling process for 20 minutes at a rotation speed of 3000 rpm.

### Step 2:

Transfer the pre-ground microalgae biomass from Step 1 into a mixing device equipped with ultrasonic heads in an inert gas environment (argon). Perform the first ultrasonication at a frequency of 40 kHz and a power of 4000 W until the biomass reaches a temperature of 45° C. Stop ultrasonication and maintain the biomass at 45° C. while stirring the mass for 40 minutes to allow the endogenous enzymes in the biomass to break down the cell walls, thereby releasing the microalgae oil. Then stop stirring and perform a second ultrasonication under the same conditions as the first one to enhance cell wall disruption and increase oil extraction efficiency.

### Step 3:

Load the biomass obtained from Step 2 into a phase separation centrifuge. Add deionized water to adjust the solid content to 10%. Operate the centrifuge at 6000 rpm to separate most of the oil from the biomass, yielding primary crude oil and primary sludge.

### Step 4:

Centrifuge the primary crude oil from Step 3 using a conical centrifuge to separate the oil phase from the aqueous phase. The aqueous phase (also called the primary liquid) contains water and valuable water-soluble compounds, and the oil phase contains microalgae oil. The centrifuge runs at 7000 rpm.

### Step 5:

Return the primary sludge from Step 3 and the primary liquid from Step 4 to the mixing device equipped with ultrasonic heads. Ultrasonicate the mixture in an inert gas environment at 40 kHz and 4000 W for 8 minutes.

### Step 6:

Load the mixture obtained from Step 5 into the phase separation centrifuge to perform secondary centrifugation. Operate the centrifuge at 6000 rpm to separate the remaining oil, yielding secondary crude oil and secondary biomass paste.

### Step 7:

Centrifuge the secondary crude oil obtained from Step 6 in the conical centrifuge to separate the oil from the aqueous phase, thereby obtaining microalgae oil and the secondary liquid (containing water and valuable water-soluble compounds). The centrifuge runs at 7000 rpm.

### Step 8:

Vacuum filter the secondary biomass paste from Step 6 to extract the liquid (referred to as vacuum liquid) from the microalgae residue.

The secondary liquid from Step 7 and the vacuum liquid from Step 8 are mixed and concentrated under vacuum to obtain a concentrated liquid containing valuable components.

Analysis of the final microalgae residue (from Step 8) shows that it contains (on a dry biomass basis): 1.1% fatty oil, 10.3% protein, 85.2% chitin- and pectin-like compounds, 3.4% other compounds, making it suitable as a raw material for agricultural, food, and industrial applications.

Condensate water from the vacuum concentration unit is recycled and reused in the phase separation centrifugation step.

The microalgae oil recovery efficiency reached 96% of the theoretical maximum.

The obtained microalgae oil has the technical specifications as presented below:

| Fatty acid composition | Content (%) |
|---|---|
| C16:0 | 14.25 |
| C16:1 | 6, .40 |
| C16:2 | 14.10 |
| C16:3 | — |
| C18:0 | 1.10 |
| C18:1 | 3, .60 |
| C18:2 | 31.21 |
| C18:3 | 22.14 |
| C20:3 | 1.04 |
| C20:5 | 1.20 |
| C22:6 | 4.96 |
| Free fatty acid content (% axit oleic): | 0.40 |
| Moisture content | 0.1 |
| Acid value (mg KOH/g) | 0.4 |
| Total omega-3 content | 28.3 |

The proportion of DHA in the total omega-3 content reaches 17.5%, demonstrating that the processing method does not degrade the valuable components in the oil. The moisture content in the oil is 0.1%, meeting the requirements for edible oils. This microalgae oil is entirely suitable for commercialization without requiring further processing.

Analysis of the composition of the obtained concentrated extract shows the presence of various valuable water-soluble bioactive compounds. Specifically, with the raw material being Chlorella sorokiniana microalgae, the concentrated extract contains nucleic acids (DNA, RNA), peptides, polysaccharides (long-chain sugars including β-glucan, rhamnan sulfate, arabinose, glucose), B-group vitamins (B1, B2, B6, B12), vitamin C, and minerals (Fe, Mg, Zn, K, Ca, P, Se, etc.). These constitute the Chlorella Growth Factor (CGF), which promotes cellular growth, enhances immunity, and supports digestion. This demonstrates that the gentle processing conditions in the patented method help retain the valuable properties of these bioactives without compromising their quality.

Given its quality, the concentrated extract product from this invention can be applied in agriculture in formulations that stimulate plant growth, enhance photosynthesis, and help plants grow rapidly and healthily. It promotes root formation, vertical growth, foliage, and flowering. It also strengthens plant immunity, enabling better resistance against diseases and pests; increases yield and quality of crops, and improves the quality of fruits, vegetables, and industrial crops.

This concentrated extract product is also suitable for use in the livestock industry to produce growth-promoting formulations for livestock, poultry, and aquaculture, enhancing nutrient absorption and supporting healthy development. It boosts animal immune systems, improving health and fertility; and increases feed efficiency, helping animals maximize nutrient uptake.

Additionally, the extract can be used in healthcare and cosmetics for health enhancement and anti-aging; it supports detoxification and body cleansing while maintaining nutritional balance. It also assists in skin cell regeneration, brightening the skin and reducing signs of aging.

The concentrated extract is also well-suited for the production of functional foods such as capsules, nutritional powders, and beverages, delivering large amounts of vitamins, minerals, amino acids, and antioxidants-thereby boosting the immune system, increasing energy, and supporting digestive health.

Furthermore, the product can be used to produce organic fertilizers, improving soil quality and providing nutrients for sustainable plant growth.

### Example 2—Application of Microalgae Residue in Hydrothermal Carbon Production

### Step 1: Loading Materials

Load the microalgae residue and deionized water into a Robinson-Mahoney basket-type catalytic reactor. The solid catalyst used is a thermally treated synthetic manganese oxide-iron oxide catalyst, shaped into cylindrical pellets and arranged in a fixed layer inside the basket, surrounding the reactor chamber. The mass ratio of microalgae residue to the volume of deionized water is 1.0:10 g/mL.

### Step 2: Hydrothermal Carbonization of Microalgae Residue

Heat the device and adjust the pressure to reach 220° C. and 2.5 MPa. Maintain these conditions for 5 hours to carry out hydrothermal carbonization of the microalgae residue, resulting in two outputs: a solid product and a liquid product (also known as hydrothermal liquor) consisting of water-soluble organic compounds. Discharge the solid product from the reactor to obtain the final product—hydrothermal carbon.

An evaluation of the Higher Heating Value (HHV) of the hydrothermal carbon made from the microalgae residue shows the HHV of the raw residue and hydrothermal carbon are 16.87 MJ/kg and 30.10 MJ/kg, respectively. The higher HHV in the hydrothermal carbon is due to its increased carbon content. This material, similar to lignite, can be used as a substitute for fossil fuels, helping reduce environmental pollution and dependency on depleting fossil resources.

### Example 3—Application of Microalgae Residue in Producing Multi-Purpose Organic Soil Conditioner

Microalgae residue, specifically Chlorella sorokiniana residue, is mixed with carbon-rich biomass to achieve a C/N ratio ranging form 27:1 to 29:1 and combined with appropriate microorganisms. The mixture is then composted in a rapid composting unit for 24 hours. The resulting material contains vital organic compounds, naturally bioactive plant-based components (vitamins, amino acids, enzymes), and the most valuable macro- and micronutrients (phosphorus, nitrogen, calcium, potassium, sulfur, iron, silver, cobalt, molybdenum, manganese, magnesium, zinc, vanadium, nickel, copper, etc.), in optimal bioavailable ratios and gently soluble forms. These enhance both plant nutrient uptake and the fertility of agricultural soil. The properties and composition of the final material can be adjusted by adding hydrothermal carbon from Example 2 or from cashew shell biochar, high-surface-area biochar, expanded vermiculite, diatomaceous earth, or peat moss. The result is a multi-purpose organic soil amendment.

This organic soil product is porous, nutrient-rich, and free from weed seeds and plant pathogens. It not only enhances but also regulates water retention, cation exchange capacity, and long-term nutrient stability. It is suitable for all types of crops, delivering special soil-derived nutrition for both potted plants and field cultivation. This product is ideal for clean, safe agricultural production systems and fits well with ecological agriculture strategies, serving as a tangible solution toward the NetZero goal.

Although the description includes several specific embodiments, those skilled in the art will recognize that other preferred embodiments can be created without departing from the scope of the invention. Therefore, the scope of the invention is defined in the attached claims.

### Achievable Benefits

As illustrated in the examples above, the patented process allows for direct lipid extraction from wet microalgae biomass, achieving high extraction efficiency and producing pure microalgae oil and valuable byproducts. The process combines bead milling for initial cell wall disruption with a kneading device equipped with ultrasonic emitters for physical cell wall breakdown. This is synergized with intracellular enzymatic autolysis during kneading under appropriate temperature and duration, enabling complete release of oil droplets and valuable compounds from microalgae cells and cell walls. Oil and valuable bioactives are then separated using primary and secondary centrifugation to improve extraction efficiency. Finally, disc-stack centrifugation separates water-soluble compounds from the oil, yielding pure microalgae oil and aqueous extract containing valuable water-soluble components, which is then concentrated.

This process is conducted at low temperatures in an inert gas environment without any added chemicals or exogenous enzymes. As a result, the oil remains pure and retains its valuable nutrients, avoiding oxidation and degradation. Furthermore, repeating the separation process twice enhances oil and bioactive extraction efficiency up to 96%. The method is low-cost, energy-efficient, and environmentally friendly since it avoids solvents, chemicals, and enzymes—making it scalable for industrial applications.

Using only wet microalgae biomass as the input under inert gas conditions, and without any chemical, solvent, or exogenous enzyme additions, this process ensures product purity and nutrient preservation, while also being environmentally friendly due to the absence of secondary waste.

Thanks to its high economic, technical, and environmental efficiency, this patented process is suitable for industrial-scale implementation.

Other features and benefits will be apparent from the description and accompanying claims.

What is claimed is:

1. A process of producing microalgae oil and valuable products from wet microalgae biomass, comprising following steps:
Step 1: load wet microalgae biomass into a bead mill and perform preliminary milling of the wet microalgae biomass;
Step 2: treat the biomass to activate intracellular enzymes to break down cell walls and microalgae cells, transfer the pre-ground biomass from Step 1 into a mixing device equipped with ultrasonic emitters in an inert gas environment, perform a first ultrasonic treatment until the biomass reaches a temperature ranging from 30° C. to 55° C., stop the ultrasound and maintain the biomass at a stable temperature, mixing it for a sufficient time to allow intrinsic enzymes to break down the cell walls, thereby facilitating the release of oil from the cells, then, stop mixing and perform a second ultrasonic treatment under the same conditions to enhance cell wall disruption and increase oil extraction efficiency;
Step 3: perform primary centrifugation on the treated biomass from Step 2 to separate liquid phase, which contains most of crude oil (still mixed with water) and water-soluble valuable compounds, from biomass residue, the separated liquid is referred to as primary crude oil, and the remaining paste is referred to as primary paste;
Step 4: centrifuge the primary crude oil obtained in Step 3 using a conical centrifuge to separate oil phase from water phase, the water phase (also known as the primary liquid) contains water and water-soluble valuable compounds, while the oil phase contains microalgae oil;
Step 5: transfer the primary paste from Step 3 and the primary liquid from Step 4 back to the mixing device with ultrasonic emitters for ultrasonic treatment of the mixture (primary paste and primary liquid) in an inert gas environment;
Step 6: perform secondary centrifugation on the ultrasonically treated mixture from Step 5 to separate the oil phase (still mixed with water) from the paste, the obtained oil (still containing water) is called secondary crude oil, and the remaining paste is called secondary paste;
Step 7: centrifuge the secondary crude oil obtained from Step 6 in a conical centrifuge to separate oil from the water phase, yielding microalgae oil and water containing valuable water-soluble compounds, referred to as secondary liquid; and
Step 8: perform vacuum filtration on the secondary paste obtained from Step 6 to extract a liquid, referred to as vacuum liquid, and collect microalgae residue, then, mix the vacuum liquid with the secondary liquid obtained in Step 7 and concentrate it under vacuum to obtain a concentrated liquid product containing valuable components.

2. The process of claim 1, wherein the wet microalgae biomass is bead milled for a duration of 5 to 20 minutes, at a milling chamber speed ranging from 1000 rpm to 3000 rpm, using zirconium silicate milling beads with a diameter ranging from 0.5 mm to 3.0 mm, and a bead-to-biomass ratio ranging from 1:1 to 2:1.

3. The process of claim 1, wherein the first and second ultrasonic treatments are conducted at a frequency ranging from 20 kHz to 40 kHz, with a power output ranging from 2000 W to 4000 W.

4. The process of claim 1, wherein the biomass is centrifuged in a phase-separation centrifuge at a speed ranging from 4000 rpm to 6000 rpm.

5. The process of claim 1, wherein the oil-water separation is performed in a conical centrifuge at a speed ranging from 5000 rpm to 7000 rpm to obtain the microalgae oil and the water phase containing water-soluble valuable compounds.

6. A process of disrupting cell walls and cells of wet microalgae biomass, comprising preliminary bead milling of biomass to initially break cell membranes, followed by a first ultrasonic treatment until the biomass reaches a temperature ranging from 30° C. to 55° C.; then mixing the ultrasonicated biomass to activate intracellular enzymes; and finally performing a second ultrasonic treatment for 2 to 8 minutes to break remaining cell walls, thereby enhancing oil recovery in subsequent steps.

7. The process of claim 6, wherein after the first ultrasonic treatment, the biomass is mixed for 20 to 50 minutes at a temperature ranging from 30° C. to 55° C.

8. The process of claim 6, wherein the wet biomass is bead milled for 5 to 20 minutes, at a chamber speed ranging from 1000 rpm to 3000 rpm, using zirconium silicate milling beads with a diameter ranging from 0.5 mm to 3.0 mm, and a bead-to-biomass ratio ranging from 1:1 to 2:1.

9. The process of claim 6, wherein the first and second ultrasonic treatments are conducted at a frequency ranging from 20 kHz to 40 kHz and a power output ranging from 2000 W to 4000 W.
